(19) United States
(12) Patent Application Publication
(10) Pub. No.: US 2017/0348285 A1
(43) Pub. Date: Dec. 7, 2017

(54) TREATMENT OF DRY EYE DISEASE WITH PARASYMPATHETIC & ANTI-SYMPATHETIC AGENTS

(71) Applicant: Harold Richard Hellstrom, Fayetteville, NY (US)

(72) Inventor: Harold Richard Hellstrom, Fayetteville, NY (US)

(21) Appl. No.: 15/607,903

(22) Filed: May 30, 2017

Related U.S. Application Data
(60) Provisional application No. 62/399,329, filed on Sep. 23, 2016.
(60) Provisional application No. 62/383,360, filed on Sep. 2, 2016.
(60) Provisional application No. 62/369,122, filed on Jul. 31, 2016.
(60) Provisional application No. 62/344,377, filed on Jun. 1, 2016.

(51) Int. Cl. A61K 31/4178; A61K 31/138; A61K 9/00; A61K 31/353; A61K 31/325

(52) U.S. Cl. CPC A61K 31/4178; A61K 31/353; A61K 31/325; A61K 9/0048; A61K 9/0051; A61K 31/138

(57) ABSTRACT

Provided are methods and combinations for treating and/or preventing dry eye disease (DED) by treating a subject in need thereof with a combination of at least one parasympathetic agent at least one anti-sympathetic effective to treat said dry eye disease.

### FIELD

Provided are methods for treating and/or preventing dry eye disease (DED) by treating a subject in need thereof with a combination of parasympathetic and anti-sympathetic agents. In particular, this combination acts by mimicking a physiological parasympathetic homeostatic shift.

### BACKGROUND

DED is a common disorder, affecting about 15-35% of the population worldwide (Xiao X, He H, Lin Z, et al. Therapeutic effects of epidermal growth factor on benzalkonium chloride-induced dry eye in a mouse model. Invest Ophthalmol Vis Sci 2012; 53:191-7). There are etiologically two types of DED (Nichols K K. The international workshop on meibomian gland dysfunction: Introduction. Invest Ophthalmol Vis Sci 2011; 52:1917-21)-the more frequent meibomian gland dysfunction (MGD) type, and the rather infrequent impaired lacrimal gland type (aqueous-deficient DED) (Lemp M A, Crews L A, Bron A J, Foulks G N, Sullivan BD. Distribution of aqueous-deficient and evaporative dry eye in a clinic-based patient cohort: A retrospective study. Cornea 2012; 31:472-8). Of 159 classifiable cases of DED, only 14% were of the pure aqueous-deficient type (Lemp M A, Crews L A, Bron A J, Foulks G N, Sullivan B D. Distribution of aqueous-deficient and evaporative dry eye in a clinic-based patient cohort: A retrospective study. Cornea 2012; 31:472-8). Patients with MGD type DED have hyperkeratinization and thick abnormal meibum.

There is a wealth of information about specific features of this disorder. However, this abundance does not appear to have coalesced into a solid understanding of basic pathogenic mechanisms for DED. It seems to be tacitly accepted that DED is due to multiple causes, and does not have one basic underlying pathogenic mechanism. In keeping with this, twenty-five “and more” causes of DED were recently listed (Mantelli F, Massaro-Giordano M, Macchi I, Lambiase A, Bonini S. The cellular mechanisms of dry eye: from pathogenesis to treatment. J Cell Physiol 2013; 228:2253-6).

Most cases of DED appear to be due to a combination of systemic and local causes. For example, there appears to be a relationship between DED and (1) rheumatoid arthritis (see, for example, (Barendregt P J, van der Heijde G L, Breedveld F C, Markusse H M. Parasympathetic dysfunction in rheumatoid arthritis patients with ocular dryness. Ann Rheum Dis 1996; 55:612-5), (2) Sjogren's syndrome (Tsifetaki N, Kitsos G, Paschides C A, et al. Oral pilocarpine for the treatment of ocular symptoms in patients with Sjogren's syndrome: A randomized 12 week controlled study. Ann Rheum Dis 2003; 62:1204-7), (3) aging (Tsubota K, Kawashima M, Inaba T, et al. The antiaging approach for the treatment of dry eye. Cornea 2012; 31:S3-S8), (4) stress, (Tsubota K, Kawashima M, Inaba T, et al. The antiaging approach for the treatment of dry eye. Cornea 2012; 31:S3-S8.; Galor A, Feuer W, Lee D J, et al. Depression, post-traumatic stress disorder, and dry eye syndrome: a study utilizing the national United States Veterans Affairs administrative database. Am J Ophthalmol 2012; 154:340-6), (5) hypertension (Uchino M, Nishiwaki Y, Michikawa T, et al. Prevalence and risk factors of dry eye disease in Japan: Koumi Study. Ophthamology 2011; 1118:2361-7), (6) myocardial infarction (Uchino M, Nishiwaki Y, Michikawa T, et al. Prevalence and risk factors of dry eye disease in Japan: Koumi Study. Ophthamology 2011; 1118:2361-7), (7) smoking (Altinors D D, Akça S, Akova YA, et al. Smoking associated with damage to the lipid layer of the ocular surface. Am J Ophthalmol 2006; 141:1016-21), (8) diabetes (Li B, Sheng M, Xie L, et al. Tear proteomic analysis of patients with type 2 diabetes and dry eye syndrome by two-dimensional nano-liquid chromatography coupled with tandem mass spectrometry. Invest Ophthalmol Vis Sci 2014; 55:177-86), (9) dyslipidemia (Jalbert I. Diet, nutraceuticals and the tear film. Exp Eye Res 2013; 117:138-46, Chun Y H, Kim H R, Han K, Park Y G, Song H J, Na K S. Total cholesterol and lipoporotein composition are associated with dry eye disease in Korean women. Lipids Health Dis 2013; 12:84), and (10) inflammation (Henrich C F, Ramulu P Y, Akpek E K. Association of dry eye and inflammatory systemic diseases in a tertiary care-baed sample. Cornea 2014; 33:819-25).

There also appears to be a relationship between DED and glaucoma. It has been found that treatment of DED in individuals with glaucoma improved intraocular pressure control (Batra R, Tailor R, Mohamed S, Ocular surface disease exacerbated glaucoma: optimizing the ocular surface improves intraocular pressure control. J Glaucoma 2014; 23:56-60).

Furthermore, there is, in fact, a higher incidence of DED/MGD with glaucoma (Lee S Y, Wong T T, Chua J, Boo C, Soh Y F, Tong L. Effect of chronic anti-glaucoma medications and trabeculectomy on tear osmolarity. Eye 2013; 27:1142-50; Viso E, Gude F, Rodriguez-Ares MT. The association of meibomian gland dysfunction and other common ocular diseases with dry eye: a population-based study in Spain. Cornea 2011; 30:1-6, Arita R, Itoh K, Maeda S, et al. Comparison of the long-term effects of various topical antiglaucoma medications on meibomian glands. Cornea 2012; 31:1229-34).

However, it is now recognized that eye drop preservatives used with glaucoma drops, such as benzalkonium chloride (Xiao X, He H, Lin Z, et al. Therapeutic effects of epidermal growth factor on benzalkonium chloride-induced dry eye in a mouse model. Invest Ophthalmol Vis Sci 2012; 53:191-7 and Lee S Y, Wong T T, Chua J, Boo C, Soh Y F, Tong L. Effect of chronic anti-glaucoma medications and trabeculectomy on tear osmolarity. Eye 2013; 27:1142-50), favor DED (Arita R, Itoh K, Maeda S, et al. Comparison of the long-term effects of various topical antiglaucoma medications on meibomian glands. Cornea 2012; 31:1229-34) and prostaglandin derivatives, which treat glaucoma, cause conjunctival hyperemia, superficial punctate keratopathy, and blepharitis (Arita R, Itoh K, Maeda S, et al. Comparison of the long-term effects of various topical antiglaucoma medications on meibomian glands. Cornea 2012; 31:1229-34). Further, various drugs used for glaucoma favor signs of MGD and superficial punctate keratopathy favors DED (Arita R, Itoh K, Maeda S, et al. Comparison of the long-term effects of various topical antiglaucoma medications on meibomian glands. Cornea 2012; 31:1229-34).

Since conventionally, DED is treated as having multiple separate mechanisms, treatment of DED can only be symptomatic (reviewed in Geerling G, Tauber J, Baudouin C, et al. The international workshop on meibomian gland dysfunction: Report of the subcommittee on management and treatment of meibomian gland dysfunction. Invest Ophthalmol Vis Sci 2011; 52:2050-64). Therapy currently includes, for example, using warm compresses and massage to liquefy and favors meibum discharge, topical anti-inflammatory agents such as cyclosporine or corticosteroids, antibiotics, and lubricating eye drops (e.g., artificial tears, see, for example, U.S. Pat. No. 4,131,651 and U.S. Pat. No. 3,947,573). Pilocarpine, a cholinergic agent used to treat glaucoma, was found to be an effective systemic treatment of dry eye disease. The topical pilocarpine formulation has been found to cause spasms of accommodation (reviewed in U.S. Pat. No. 6,277,855). However, recently, low dose topical pilocarpine (0.05%) has been found to stimulate tear production without any change in pupil size after treatment for two months (Urriquia T B and Marin, Jr. JDF. Efficacy of Topical Pilocarpine in the Management of Primary Aqueous Tear Deficiency: An Initial Study. Philip J Ophthalmol 2014; 39:6-11).

Other treatments proposed involve the use of acetylcholinesterase (AchE) inhibitors (U.S. Pat. No. 6,273,092), nicotinic acetylcholine receptor agonists (U.S. Pat. No. 6,277,885), neurotransmitter such as acetylcholine, ATP, glycine, glutamate, dopamine, norepinephrine, epinephrine, octopamine, serotonin (5-hydroxytryptamine), beta-alanine, histamine, gamma aminobutyric acid (GABA), taurine, aspartate and nitric oxide and neuropeptides (US Pat. Appln. Pub. Nos. 20060270592 and 20080261890).

### SUMMARY

Provided are methods and compositions for treating and/or preventing dry eye disease to a subject in need thereof. In a particular embodiment, said subject may be a mammal (e.g., dog, cat, human); in an even more particular embodiment, said subject may be a human patient. The method comprises administering an amount of at least one parasympathomimetic agent and at least one anti-sympathetic agent effective to treat said dry eye disease. In a particular embodiment, the subject is administered said parasympathomimetic agent and/or anti-sympathetic agent in an amount at least about 10% below the known effective dosage to treat glaucoma or other eye diseases. In a more particular embodiment, the subject is administered at least about 50% to about 80% below the dosage of said parasympathomimetic agent and/or anti-sympathetic agent. In yet another particular embodiment, the agents are administered topically and thus the composition is a topical composition.

In a particular embodiment, said DED is contact lens induced DED. In a related aspect, said parasympathetic agent and sympathetic agent are applied to the contact lens. In yet another related aspect, provided is a method for preventing and/or treating DED, in particular, contact lens induced DED, in a subject in need thereof comprising applying to said subject a contact lens comprising an amount of at least one parasympathomimetic agent and optionally at least one anti-sympathetic agent effective to treat said DED, in particular, contact lens induced DED. In yet another related aspect, provided is a contact lens comprising an amount of at least one parasympathomimetic agent and at least one anti-sympathetic agent effective to treat and/or prevent DED, in particular, contact lens induced DED. Also provided is a method for producing said contact lens comprising applying amounts of at least one parasympathomimetic agent and optionally at least one anti-sympathetic agent effective to treat and/or prevent DED.

In a more particular embodiment, the method and compositions act by mimicking a homeostatic parasympathetic shift. The basic principles of a parasympathetic homeostatic shift can be found in Hellstrom HR. The altered homeostatic theory: A hypothesis proposed to be useful in preventing ischemic heart disease, hypertension, and diabetes—including reducing the risk of age and atherosclerosis. Med Hypotheses. 2007; 68:415-433. This altered homeostatic theory argues that risk factors favor a disadvantageous sympathetic homeostatic shift and disease, and that preventative factors favor a beneficial parasympathetic homeostatic shift and health. As an example, the multiple risk factors listed above are regarded as favoring DED through a disadvantageous sympathetic homeostatic shift, and treatment of DED used in this invention favors a beneficial parasympathetic homeostatic shift and improvement of DED. A beneficial parasympathetic homeostatic shift includes both increased parasympathetic activity and decreased sympathetic activity. Thus, a combination of parasympathomimetic and anti-sympathetic agents are used.

In a particular embodiment, the anti-sympathetic agent or agents is a beta-blocker and may include but is not limited to niprodilol, nebivolol, propranolol and more particularly, niprodilol and nebivolol. In another particular embodiment, the parasympathomimetic agent or agents is selected from the group consisting of pilocarpine and carbachol. In a specific embodiment, the subject may be administered a combination or composition comprising (a) carbachol and (b) niprodilol and/or nebivolol and/or propranolol.

In yet another particular embodiment, also provided are angiotensin blockers (angiotensin-converting enzyme (ACE) inhibitors and angiotensin II receptor blockers). These agents may be regarded as anti-sympathetic agents since they block the renin-angiotensin-aldosterone system (Slomka T, Lennon E S, Akbar H, et al. Effects of Renin-Angiotensin-Aldosterone System Blockade in Patients with End-Stage Renal Disease. Am J Med Sci 2016; 351:309-16). Specifically, ACE inhibitors (Hirooka K, Shiraga F. Potential role for angiotensin-converating enzyme inhibitors in the treatment of glaucoma. Clin Ophthalmol 2007; 1:217-23) and angiotensin II receptor blockers (Burnier M. Angiotensin II receptor antagonists. Lancet 2000; 355:637-45) block angiotensin II—and angiotensin II increases sympathetic activation (Reid I A. Interactions between ANG II, sympathetic nervous system, and baroreceptor reflexes in regulation of blood pressure. Am J Physiol 1992; 262:E63-E78). Thus, angiotensin blockers reduce sympathetic activation and additionally prompt parasympathetic activation (Hellstrom H R. The altered homeostatic theory: a hypothesis proposed to be useful in understanding and preventing ischemic heart disease, hypertension, and diabetes—including reducing the risk of age and atherosclerosis. Med Hypotheses 2007; 68:415-33). It should be noted that renin-angiotensin regulation is present in the eye (Hirooka K, Shiraga F. Potential role for angiotensin-converating enzyme inhibitors in the treatment of glaucoma. Clin Ophthalmol 2007; 1:217-23). Also, ACE inhibitors have been found to be associated with a lower risk of DED (Anonymous. American Academy of Ophthalmology Cornea/External Disease Panel. Preferred Practice Pattern® Guidelines. Dry Eye Syndrome. American Academy of Ophthalmology. 2013. http://one.aao.org/preferred-practice-pattern/dry-eye-syndrome-ppp-2013. Accessed Feb. 15, 2015. Approved Sep. 21, 2013) and prompt parasympathetic activation. ACE inhibitors have also been found to improve diabetic retinopathy (Chaturvedi N, Sjolie A K, Stephenson J M, et al. Effect of lisinopril on progression of retinopathy in normotensive people with type 1 diabetes. The EUCLID Study Group. EURODIAB Controlled Trial of Lisinopril in Insulin-Dependent Diabetes Mellitus. Lancet 1998; 351:28-31), and lowered intraocular pressure in glaucoma (Hirooka K, Shiraga F. Potential role for angiotensin-converating enzyme inhibitors in the treatment of glaucoma. Clin Ophthalmol 2007; 1:217-23).

As previously discussed in more detail, the amounts of parasympathomimetic and/or anti-sympathetic agents are at least about 10% less than the amounts used to treat glaucoma or other eye disorders. Niprovilol (Mizuno K I, Koide T, Yoshimura M, Araie M. Neuroprotective effect and intraocular penetration of nipradilol, a beta-blocker with nitric oxide donative action Invest Ophth Vis Sci. 2001; 42:688-92) and nebivolol (Zhang A, Ding L, Jin Z, et al. Nebivolol protects against myocardial infarction injury via stimulation of beta 3-adrenergic receptors and nitric oxide signaling. PlosONE. 2014; 9:e98179) may be used in a particular embodiment, as they have a healing nitric oxide (NO) donative action. This is pertinent as the functional lacrimal unit includes a nitrergic innervation (Bolekova A, Kluchov D, Tomasova L, Hvizdosova N. Effect of retinoic acid on the nitrergic innervation of meibomian glands in rats. Eur J Histochem. 2012; 56:e50).

### Definitions

Where a range of values are provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this disclosure are incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

It must be noted that as used herein and in the appended claims, the singular forms “a,” “and” and “the” include plural references unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention. Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. Thus the terms “comprising”, “including,” containing”, “having” etc. shall be read expansively or open-ended and without limitation. When used herein, the term “comprising” can be substituted with the term “containing” or sometimes when used herein with the term “having”.

As defined herein, the terms “dry eye”, “dry eye disease” or “dry eye syndrome”, means any disease or disorder or condition which results in an adverse effect on the functional lacrimal unit, i.e. on the quality of the tear film that lubricates the eyes The disease or disorder may be of the eye itself, or of another part of the body, so long as it results in an adverse effect on the quality of the tear film that lubricates the eyes. For example, “dry eye” as used herein includes dry eye disorder, Riley Day Syndrome and keratoconjunctivities sicca, as well as dry eye caused by other conditions, factors and phenomena such as diabetes, prolonged contact lens wear, advanced age, circulating hormones, various autoimmune diseases (e.g. Sjorgren's syndrome and systemic lupus erythematosus), ocular surgeries including PRK or LASIK, many medications, environmental conditions, visual tasking such as computer use, ocular fatigue, mechanical influences such as corneal sensitivity, partial lid closure, surface irregularities (e.g. pterygium), and lid irregularities (e.g. ptosis, entropion/ectropion, pinguecula).

The phrases “effective amount” or “amount effective” are art-recognized terms, and refer to an amount of an agent that, when incorporated into a compositions set forth herein, produces some desired effect at a reasonable benefit/risk ratio applicable to any medical treatment. In certain embodiments, the term refers to that amount necessary or sufficient to eliminate, reduce or maintain (e.g., prevent the spread of) a symptom of dry eye, or prevent or treat dry eye. The effective amount may vary depending on such factors as the disease or condition being treated, the particular composition being administered, or the severity of the disease or condition. One of skill in the art may empirically determine the effective amount of a particular agent without necessitating undue experimentation.

As defined herein, a “physiological parasympathetic homeostatic shift” means to shift homeostasis toward parasympathetic dominance where there is an increase in parasympathetic activity and a corresponding decrease in sympathetic activity.

As defined herein, the terms “treat”, “treatment” and “treating” are to be understood accordingly as embracing prophylaxis and treatment or amelioration of symptoms of disease as well as treatment of the cause of the disease.

### DETAILED DESCRIPTION

As set forth above, provided are methods for administering and compositions for preventing and/or treating dry eye disease comprising one or more parasympathomimetic agents and at least one anti-sympathetic agent in amounts effective to prevent and/or treat said dry eye disease. Also provided is the use of at least one parasympathomimetic and at least one anti-sympathetic agent in amounts effective to prevent and/or treat dry eye disease and/or formulate a medicament for the prevention and/or treatment of dry eye disease.

### Parasympathomimetic Agents

There are multiple parasympathomimetic agents, and several are formulated for topical use. These include but are not limited to pilocarpine, carbachol, ecothiopate, demecarium bromide and diisopropyl fluorophosphate mentioned. In a specific embodiment, the parasympathomimetic agent may be carbachol, echothiophate iodide, physostigmine and/or demecarium bromide. More than one parasympathomimetic agent may be used to secure a wider spectrum of results.

### Sympathetic Blockers/Anti-Sympathetic Agents

Sympathetic blockers are either alpha or beta selective. Alpha blockers probably are not helpful. As an example, the alpha blocker tamsulosin can cause the floppy iris syndrome during eye surgery (Chang D F, Campbell J R, Colin J, Schweitzer C. Prospective masked comparison of intraoperative floppy iris syndrome severity with tamsulosin versus alfuzosin. Ophthalmology 2014; 121:829-34) and also can cause retrograde ejaculation (Agrawal M, Gupta M, Gupta A et al. Prospective randomized trial comparing efficacy of alfuzosin and tamsulosin in management of lower ereteral stones. Urology 2009; 73:706-9). However, to ensure a more physiological blocking of sympathetic activation, an alpha blocker, probably at rather low doses, might be used.

In another specific embodiment, the sympathetic blocker or anti-sympathetic agent may be a beta-blocker. The beta-blocker may be a beta-selective or non-selective agent and may include but is not limited to propanolol (nonselective) timolol (nonselective), betaxolol (beta 1 selective antagonist), levobunolol (nonselective beta 1 and 2 blocking agent), carteolol (nonselective beta-blocker), metipranolol (nonselective beta-blocker) levobetaxolol (beta 1 inhibitor) as well as nitric oxide (NO) donors such as nipradilol (nonselectable beta-blocker) and nebivolol (betal selective blocker) and additionally, substances having ISA or intrinsic sympathetic action such as oxyprenolol and pindolol.

### Dose Levels

As noted above in more detail, in one embodiment, at least one of the agents set forth above is administered at a lower dose than of any known therapeutic dose level for glaucoma or other eye disease. In a particular embodiment, at least one of the agents is administered at a dose at least about 10% lower than that used to treat glaucoma or other eye diseases. In an even more particular embodiment, at least one of said agents are administered at a dose of at least about 50% to about 80% lower than the known effective dose used to treat glaucoma or other eye diseases and in even yet a more particular embodiment, said agents are administered at a dose of at least about 40% to about 25% lower than the known effective dose used to treat glaucoma or other eye diseases.

The agents set forth above may be administered once, twice or three times a day. In one embodiment, the two agents would be administered in one formulation or together. In a particular embodiment, the two agents are administered in one formulation.

In a specific embodiment, carbachol, a parasympathomimetic agent, which stimulates both muscarinic and nicotinic receptors, may be administered at around 0.25% w/v to about 0.5% w/v and nipradilol, a beta-blocker with nitroglycerin-like vasodilative activities may be administered at a dose of about 0.05% to about 0.1% w/v. Alternately, the beta blocker, carteolol, which has some intrinsic sympathomimetic activity may be used. It may be administered at a dose of between about 0.25% w/v to about 0.35% w/v.

### Angiotensin Blockers

Also provided are angiotensin blockers which include but are not limited to ACE inhibitors and angiotensin II receptor blockers. Angiotensin blockers may, as noted above, may act as anti-sympathetic agents as well as parasympathomimetic agents. Examples of ACE inhibitors include but are not limited to benazepril, captopril, enalapril, fosinopril, lisinopril, moexipril, perindopril, quinapril, ramipril and trandolapril. Examples of angiotensin ll receptor antagonists include but are not limited to Losartan, Valsartan, Irbesartan, Candesartan, Telmisartan, Eprosartan.

### Compositions/Formulation

In certain embodiments, the composition comprises at least one parasympathomimetic and at least one anti-sympathetic agent. Preferably, the compositions will be formulated as solutions, suspensions and other dosage forms for topical ophthalmic administration in a pharmaceutically acceptable carrier, adjuvant, or vehicle. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semi-solid compositions.

Any of a variety of carriers may be used in the topical formulations including water, mixtures of water and water-miscible solvents, such as C1- to C7-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carpool, or mixtures of those polymers. The concentration of the carrier is, typically, from 1 to 100,000 times the concentration of the active ingredient.

Additional ingredients that may be included in the formulations include tonicity enhancers, preservatives, solubilizers, non-toxic excipients, demulcents, sequestering agents, pH adjusting agents, co-solvents and viscosity building agents.

For the adjustment of the pH, preferably to a physiological pH, buffers may especially be useful. The pH of the present solutions should be maintained within the range of 4.0 to 8.0, more preferably about 4.0 to 6.0, more preferably about 6.5 to 7.8. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Borate buffers are preferred. Generally, buffers will be used in amounts ranging from about 0.05 to 2.5 percent by weight, and preferably, from 0.1 to 1.5 percent.

Tonicity is adjusted, if needed, typically by tonicity enhancing agents. Such agents may, for example, be of ionic and/or non-ionic type. Examples of ionic tonicity enhancers are alkali metal or earth metal halides, such as, for example, CaCl₂, KBr, KCl, LiCl, NaI, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids which is equivalent to a 0.9% solution of sodium chloride or a 2.5% solution of glycerol. An osmolality of about 225 to 400 mOsm/kg is preferred, more preferably 280 to 320 mOsm.

In certain embodiments, the topical formulations additionally comprise a preservative. A preservative may typically be selected from a quaternary ammonium compound such as benzalkonium chloride, benzoxonium chloride or the like. Benzalkonium chloride is better described as: N-benzyl-N-(C8-C18 alkyl)-N,N-dimethylammonium chloride. Examples of preservatives different from quaternary ammonium salts are alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal® or sorbic acid. Preferred preservatives are quaternary ammonium compounds, in particular, benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In another embodiment, the topical formulations do not include a preservative. Such formulations would be useful for patients who wear contact lenses, or those who use several topical ophthalmic drops and/or those with an already compromised ocular surface wherein limiting exposure to a preservative may be more desirable.

In a particular embodiment, said parasympathetic agent(s) and sympathetic agent(s) may be incorporated, attached or carried on a contact lens using procedures known in the art. In a particular embodiment for preventing and/or treating DED, in particular, contact lens-induced DED, the invention includes loading parasympathomimetic and anti-sympathetic agents into or onto drug-eluting contact lenses, as hydrogel contact lenses, at doses appropriate for preventing and treating DED, in particular, contact lens induced DED. As an example of drug loading of drug-eluting contact lenses, as hydrogel contact lenses, a preliminary study for the treatment of glaucoma uses the beta-blocker timolol loaded into drug-eluting contact lenses, such as

silicone-hydrogel contact lenses (see, for example, Jung H J, Abou-Jaoude M, Carbia B E, Plummer C, Chauhan A. Glaucoma therapy by extended release of timolol from nanoparticle loaded silicone-hydrogel contact lenses. J Controlled Release 2013; 165:82-9). Of interest, in another preliminary study of drug-eluting contact lenses, two agents were used for the purpose of treating glaucoma. (Hsu K H, Carbia B E, Plummer C, Chauhan A. Dual drug delivery from vitamin E loaded contact lenses for glaucoma therapy. Eur J Pharmaceut Biopharmaceut 2015; 94:312-21).

The topical formulation may additionally require the presence of a solubilizer, in particular if the active or the inactive ingredients tends to form a suspension or an emulsion. A solubilizer suitable for an above concerned composition, is for example, selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of a solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements and is generally in the range of from approximately 0.0001 to approximately 90% by weight.

Other compounds may also be added to the formulations of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family and vinyl polymers. In other embodiments, the pharmaceutical compositions according to the present invention will be formulated for other types of administration, such as oral, parenteral, inhalation spray, nasal, buccal, or via an implanted reservoir. The term parenteral as used herein includes subcutaneous, intracutaneous, intravenous, intramuscular, intra articular, intrasynovial, intrasternal, intrathecal, intralesional, and intracranial injection or infusion techniques. Methods of formulating pharmaceutical compositions for such forms of administration are well-known to one of skill in the art.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a molecule thereof as an active ingredient. Compositions of the present invention may also be administered as a bolus, electuary, or paste.

In solid dosage forms for oral administration (capsules, tablets, pills, dragees, powders, granules and the like), the particle is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium molecules; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the supplement or components thereof moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the compound, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in p articular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

What is claimed is:

1. A method for treating and/or preventing dry eye disease in a subject in need thereof comprising administering an amount of at least one parasympathomimetic agent and at least one anti-sympathetic agent effective to treat said dry eye disease.

2. The method according to claim 1, wherein said agents are administered topically.

3. The method according to claim 1, wherein said subject is administered said parasympathomimetic agent and/or anti-sympathetic agent in an amount at least 10% below known effective dosage to treat glaucoma or other eye diseases.

4. The method according to claim 1, wherein said anti-sympathetic agent is a beta-blocker.

5. The method according to claim 1, wherein said parasympathomimetic agent is selected from the group consisting of pilocarpine and carbachol.

6. The method according to claim 4, wherein said beta-blocker is selected from the group consisting of niprodilol, nebivolol, propranolol.

7. The method according to claim 1 wherein said method acts by mimicking a physiological homeostatic parasympathetic shift.

8. The method according to claim 1, wherein said parasympathomimetic agent and anti-sympathetic agent are administered in the form of a composition.

9. A composition or combination for treating and/or preventing dry eye disease to a subject in need thereof comprising an amount of at least one parasympathomimetic agent and at least one anti-sympathetic agent effective to treat said dry eye disease.

10. The method according to claim 1, wherein said DED is contact lens induced DED.

11. The method according to claim 1, wherein said agents are applied to contact lenses.

12. The method according to claim 8, wherein said composition is in the form of drops or in the form of contact lenses comprising at least one parasympathomimetic agent and at least one anti-sympathetic agent.

13. A method for preventing and/or treating contact lens induced DED in a subject in need thereof comprising inserting a contact lens comprising an amount of at least one parasympathomimetic agent and optionally at least one anti-sympathetic agent effective to treat said contact lens induced DED.

14. A contact lens comprising an amount of at least one parasympathomimetic agent and at least one anti-sympathetic agent effective to treat and/or prevent DED.

15. The contact lens according to claim 14, wherein said DED is contact lens induced DED.

16. A method for producing the contact lens of claim 14, comprising applying amounts of at least one parasympathomimetic agent and optionally at least one anti-sympathetic agent effective to treat and/or prevent DED.

17. The method according to claim 1 wherein said parasympathetic and/or anti-sympathetic agent is an angiotensin blocker.

18. The method according to claim 13 wherein said parasympathetic and/or anti-sympathetic agent is an angiotensin blocker.

19. The composition or combination according to claim 9 wherein said parasympathetic and/or anti-sympathetic agent is an angiotensin blocker.

20. The contact lens according to claim 14 wherein said parasympathetic and/or anti-sympathetic agent is an angiotensin blocker.
